SEQUENCE LISTING (6 sequences)

SEQ ID NO: 1 - protein, 3256 aa, Homo sapiens
MWPTRRLVTIKRSGVDGPHFPLSLSTCLFGRGIECDIRIQLPVVSKQHCKIEIHEQEAILHNFSSTNPTQVNGSVIDEPVRLKHGDVITIIDRSFRYENESLQNGRKSTEFPRKIREQEPARRVSRSSFSSDPDEKAQDSKAYSKITEGKVSGNPQVHIKNVKEDSTADDSKDSVAQGTTNVHSSEHAGRNGRNAADPISGDFKEISSVKLVSRYGELKSVPTTQCLDNSKKNESPFWKLYESVKKELDVKSQKENVLQYCRKSGLQTDYATEKESADGLQGETQLLVSRKSRPKSGGSGHAVAEPASPEQELDQNKGKGRDVESVQTPSKAVGASFPLYEPAKMKTPVQYSQQQNSPQKHKNKDLYTTGRRESVNLGKSEGFKAGDKTLTPRKLSTRNRTPAKVEDAADSATKPENLSSKTRGSIPTDVEVLPTETEIHNEPFLTLWLTQVERKIQKDSLSKPEKLGTTAGQMCSGLPGLSSVDINNFGDSINESEGIPLKRRRVSFGGHLRPELFDENLPPNTPLKRGEAPTKRKSLVMHTPPVLKKIIKEQPQPSGKQESGSEIHVEVKAQSLVISPPAPSPRKTPVASDQRRRSCKTAPASSSKSQTEVPKRGGRKSGNLPSKRVSISRSQHDILQMICSKRRSGASEANLIVAKSWADVVKLGAKQTQTKVIKHGPQRSMNKRQRRPATPKKPVGEVHSQFSTGHANSPCTIIIGKAHTEKVHVPARPYRVLNNFISNQKMDFKEDLSGIAEMFKTPVKEQPQLTSTCHIAISNSENLLGKQFQGTDSGEEPLLPTSESFGGNVFFSAQNAAKQPSDKCSASPPLRRQCIRENGNVAKTPRNTYKMTSLETKTSDTETEPSKTVSTANRSGRSTEFRNIQKLPVESKSEETNTEIVECILKRGQKATLLQQRREGEMKEIERPFETYKENIELKENDEKMKAMKRSRTWGQKCAPMSDLTDLKSLPDTELMKDTARGQNLLQTQDHAKAPKSEKGKITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTPGPSEESMTDEKTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTPGHTEELVAAGKTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTPGHTEEAVAAGKTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGLKELFQTPVCTDKPTTHEKTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQTRGHTEESMTNDKTAKVACKSSQPDPDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTPSHTKESMTNEKTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTPCTDNPTTDEKTTKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTPGHTEESMTDDKITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTPDHTEESTTDDKTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTPICTDKPTTHEKTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTPGTDKPTTDEKTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTPGHTEESMTDDKITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAPGHTEESMTIDKNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSETSGHTQESLTAGKATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKDPAAGHTEESMTDDKTTKIPCKSSPELEDTATSSKRRPRTRAQKVEVKEELLAVGKLTQTSGETTHTDKEPVGEGKGTKAFKQPAKRKLDAEDVIGSRRQPRAPKEKAQPLEDLASFQELSQTPGHTEELANGAADSFTSAPKQTPDSGKPLKISRRVLRAPKVEPVGDVVSTRDPVKSQSKSNTSLPPLPFKRGGGKDGSVTGTKRLRCMPAPEEIVEELPASKKQRVAPRARGKSSEPVVIMKRSLRTSAKRIEPAEELNSNDMKTNKEEHKLQDSVPENKGISLRSRRQNKTEAEQQITEVFVLAERIEINRNEKKPMKTSPEMDIQNPDDGARKPIPRDKVTENKRCLRSARQNESSQPKVAEESGGQKSAKVLMQNQKGKGEAGNSDSMCLRSRKTKSQPAASTLESKSVQRVTRSVKRCAENPKKAEDNVCVKKIRTRSHRDSEDI

SEQ ID NO: 2 - DNA, 12507 bp, Homo sapiens
taccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaaattgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagaaagctcaagattccaaggcctattcaaaaatcactgaaggaaaagtttcaggaaatcctcaggtacatatcaagaatgtcaaagaagacagtaccgcagatgactcaaaagacagtgttgctcagggaacaactaatgttcattcctcagaacatgctggacgtaatggcagaaatgcagctgatcccatttctggggattttaaagaaatttccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatgaagacccctgtacaatattcacagcaacaaaattctccacaaaaacataagaacaaagacctgtatactactggtagaagagaatctgtgaatctgggtaaaagtgaaggcttcaaggctggtgataaaactcttactcccaggaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagccatttttaactctgtggctcactcaagttgagaggaagatccaaaaggattccctcagcaagcctgagaaattgggcactacagctggacagatgtgctctgggttacctggtcttagttcagttgatatcaacaactttggtgattccattaatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggagggagaaagagtggcaacctgccttcaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaaaataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataagaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctccagggcacggtggcaggaacaactatcctcgtctgtcccaacactgagcaggcactcggtaaacacgaatgaatggatgagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgactgcatttcctggttactctatcccagtgaccagccacaggagatgtccaataaagtatgtgatgaaatggtcttaaaaaaaaaaaaaa

SEQ ID NO: 3 - protein, 2896 aa, Homo sapiens
MWPTRRLVTIKRSGVDGPHFPLSLSTCLFGRGIECDIRIQLPVVSKQHCKIEIHEQEAILHNFSSTNPTQVNGSVIDEPVRLKHGDVITIIDRSFRYENESLQNGRKSTEFPRKIREQEPARRVSRSSFSSDPDESEGIPLKRRRVSFGGHLRPELFDENLPPNTPLKRGEAPTKRKSLVMHTPPVLKKIIKEQPQPSGKQESGSEIHVEVKAQSLVISPPAPSPRKTPVASDQRRRSCKTAPASSSKSQTEVPKRGGRKSGNLPSKRVSISRSQHDILQMICSKRRSGASEANLIVAKSWADVVKLGAKQTQTKVIKHGPQRSMNKRQRRPATPKKPVGEVHSQFSTGHANSPCTIIIGKAHTEKVHVPARPYRVLNNFISNQKMDFKEDLSGIAEMFKTPVKEQPQLTSTCHIAISNSENLLGKQFQGTDSGEEPLLPTSESFGGNVFFSAQNAAKQPSDKCSASPPLRRQCIRENGNVAKTPRNTYKMTSLETKTSDTETEPSKTVSTANRSGRSTEFRNIQKLPVESKSEETNTEIVECILKRGQKATLLQQRREGEMKEIERPFETYKENIELKENDEKMKAMKRSRTWGQKCAPMSDLTDLKSLPDTELMKDTARGQNLLQTQDHAKAPKSEKGKITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTPGPSEESMTDEKTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTPGHTEELVAAGKTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTPGHTEEAVAAGKTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGLKELFQTPVCTDKPTTHEKTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQTRGHTEESMTNDKTAKVACKSSQPDPDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTPSHTKESMTNEKTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTPCTDNPTTDEKTTKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTPGHTEESMTDDKITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTPDHTEESTTDDKTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTPICTDKPTTHEKTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTPGTDKPTTDEKTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTPGHTEESMTDDKITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAPGHTEESMTIDKNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSETSGHTQESLTAGKATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKDPAAGHTEESMTDDKTTKIPCKSSPELEDTATSSKRRPRTRAQKVEVKEELLAVGKLTQTSGETTHTDKEPVGEGKGTKAFKQPAKRKLDAEDVIGSRRQPRAPKEKAQPLEDLASFQELSQTPGHTEELANGAADSFTSAPKQTPDSGKPLKISRRVLRAPKVEPVGDVVSTRDPVKSQSKSNTSLPPLPFKRGGGKDGSVTGTKRLRCMPAPEEIVEELPASKKQRVAPRARGKSSEPVVIMKRSLRTSAKRIEPAEELNSNDMKTNKEEHKLQDSVPENKGISLRSRRQNKTEAEQQITEVFVLAERIEINRNEKKPMKTSPEMDIQNPDDGARKPIPRDKVTENKRCLRSARQNESSQPKVAEESGGQKSAKVLMQNQKGKGEAGNSDSMCLRSRKTKSQPAASTLESKSVQRVTRSVKRCAENPKKAEDNVCVKKIRTRSHRDSEDI

SEQ ID NO: 4 - DNA, 11427 bp, Homo sapiens
taccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaaattgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggagggagaaagagtggcaacctgccttcaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaaaataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataagaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctccagggcacggtggcaggaacaactatcctcgtctgtcccaacactgagcaggcactcggtaaacacgaatgaatggatgagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgactgcatttcctggttactctatcccagtgaccagccacaggagatgtccaataaagtatgtgatgaaatggtcttaaaaaaaaaaaaaa

SEQ ID NO: 5 - protein, 604 aa, Homo sapiens [sequence omitted]

SEQ ID NO: 6 - DNA, 3387 bp, Homo sapiens
gtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtgggaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgagggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataaggacgaaagtaaagatgtttgaatacttaaacactatcacaagatgccaaaatgctgaaagtttttacactgtcgatgtttccaatgcatcttccatgatgcattagaagtaactaatgtttgaaattttaaagtacttttgggtatttttctgtcatcaaacaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatatcctgttgagatattccagaatctgtttatatggctggtaacatgtaaaaaccccataaccccgccaaaaggggtcctacccttgaacataaagcaataaccaaaggagaaaagcccaaattattggttccaaatttagggtttaaactttttgaagcaaacttttttttagccttgtgcactgcagacctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcaaacacttcgtttctcctattttgttttactagttttaagatcagagttcactttctttggactctgcctatattttcttacctgaacttttgcaagttttcaggtaaacctcagctcaggactgctatttagctcctcttaagaagattaaaaaaaaaaaaaaag

(19) United States
(12) Patent Application Publication
(10) Pub. No.: US 2011/0136129 A1
(43) Pub. Date: Jun. 9, 2011

(54) ASSESSING ATYPICAL HYPERPLASIA PATIENTS FOR THE RISK OF DEVELOPING BREAST CANCER

(76) Inventors: Lynn C. Hartmann, Rochester, MN (US); Marlene Frost, Burnsville, MN (US); Carol Reynolds, Rochester, MN (US); Vernon Pankratz, Rochester, MN (US)

(21) Appl. No.: 12/960,894

(22) Filed: Dec. 6, 2010

Related U.S. Application Data
(60) Provisional application No. 61/285,062, filed on Dec. 9, 2009.

(51) Int. Cl. C12Q 1/68

(52) U.S. Cl. 435/6.14

(57) ABSTRACT

This document provides methods and materials involved in assessing the risk of developing breast cancer in patients with atypical hyperplasia. For example, methods and materials for using the level of Ki67 expression (e.g., percent Ki67⁺cells or Ki67 staining intensity) to determine if a patient with atypical hyperplasia has an increased risk of developing breast cancer within an early phase from diagnosis (e.g., within ten years of biopsy) or an increased risk of developing breast cancer at a late phase from diagnosis (e.g., at a point more than ten years of biopsy) are provided.

### CROSS RELATED APPLICATIONS

This application claims the benefit of U.S. provisional application 61/285,062, filed Dec. 9, 2009. The disclosure of the prior application is considered part of (and is incorporated by reference in) the disclosure of this application.

### BACKGROUND

1. Technical Field

This document relates to methods and materials involved in assessing the risk of developing breast cancer in patients with atypical hyperplasia. For example, this document relates to methods and materials for using the level of Ki67 expression (e.g., percent Ki67⁺cells or Ki67 staining intensity) to determine if a patient with atypical hyperplasia has an increased risk of developing breast cancer and the time course of that risk (e.g., within ten years of atypia biopsy or an increased risk of developing breast cancer at a point more than ten years after biopsy).

2. Background Information

Women with atypical hyperplasia have a four fold increased risk for breast cancer compared to women in the general population with a cumulative incidence approaching 30% at 25 years. Because of their high-risk status, women with atypia are frequently counseled to follow heightened surveillance and to take tamoxifen for chemoprevention; some even consider prophylactic mastectomy.

A frequently used model for risk prediction in women with atypia is the Gail model, which appears to lack sufficient accuracy in predicting risk of individual women with atypia (Pankratz et al., J. Clin. Oncol., 26:5374-9 (2008)). From a biologic standpoint, atypical hyperplasia is recognized as a precursor lesion to both non-invasive and invasive breast cancer.

### SUMMARY

This document provides methods and materials for assessing the risk of developing breast cancer in patients with atypical hyperplasia. For example, this document provides methods and materials for using the level of Ki67 polypeptide expression in an atypical hyperplasia sample to determine if the patient with atypical hyperplasia has an increased risk of developing breast cancer within an early phase from diagnosis (e.g., within ten years of biopsy or diagnosis) or an increased risk of developing breast cancer at a late phase from diagnosis (e.g., at a time point more than ten years after biopsy or diagnosis). Determining if a human patient has either (a) an increased risk of developing breast cancer within the first ten years of biopsy or diagnosis or (b) an increased risk of developing breast cancer at some time point after the first ten years of biopsy or diagnosis can allow physicians and the patient to determine a course of treatment or monitoring appropriate for that patient. For example, a patient found to have a reduced risk of developing breast cancer during the first ten years after being diagnosed with atypical hyperplasia and an increased risk of developing breast cancer after ten years of being diagnosed can elect to forgo risk reducing strategies such as chemoprevention or prophylactic mastectomy until she approaches the ten year point from diagnosis. Likewise, a patient found to have an increased risk of developing breast cancer within the first ten years of being diagnosed with atypia can elect to undergo more frequent monitoring, chemoprevention, and/or a prophylactic mastectomy at a time point closer to the initial diagnosis (e.g., within one, two, three, four, or five years of being diagnosed with atypical hyperplasia).

In general, one aspect of this document features a method for determining an atypical hyperplasia patient's time period of increased risk for developing breast cancer. The method comprises, or consists essentially of, determining if an atypical hyperplasia sample obtained from a patient contains an increased level of Ki67 or COX-2 expression, classifying the patient as having an increased risk of developing breast cancer within an early phase from diagnosis if the atypical hyperplasia sample contains the increased level of Ki67 or COX-2 expression, and classifying the patient as having an increased risk of developing breast cancer within a late phase from diagnosis if the atypical hyperplasia sample does not contain the increased level of Ki67 or COX-2 expression. The sample can be a tissue section. The method can comprise determining if the sample contains an increased level of Ki67 expression. The increased level of Ki67 expression can be greater than 2.0 percent Ki67⁺cells within the sample. The method can comprise determining if the sample contains an increased level of COX-2 expression. The method can comprise determining if the sample contains an increased level of Ki67 or COX-2 expression using an anti-Ki67 antibody or an anti-COX-2 antibody. The method can comprise determining if the sample contains an increased level of Ki67 or COX-2 expression using a nucleic acid detection method. The early phase from diagnosis can be within eight years of diagnosis. The early phase from diagnosis can be within nine years of diagnosis. The early phase from diagnosis can be within ten years of diagnosis. The late phase from diagnosis can be the time after ten years from diagnosis.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 contains photographs of examples of tissue having low (A) and high (B) Ki67 staining values. 0.14% cells positive for Ki67 in FIG. 1A, while 9.7% cells positive for Ki67 in FIG. 1B.

FIG. 2 is a graph plotting the observed and expected cumulative breast cancer incidence as a function of follow-up interval, stratified by Ki67 staining levels.

FIG. 3 contains the amino acid sequence of a human Ki67 polypeptide (SEQ ID NO:1).

FIG. 4 contains the nucleic acid sequence that encodes a human Ki67 polypeptide (SEQ ID NO:2).

FIG. 5 contains the amino acid sequence of a human Ki67 polypeptide (SEQ ID NO:3).

FIG. 6 contains the nucleic acid sequence that encodes a human Ki67 polypeptide (SEQ ID NO:4).

FIG. 7 contains the amino acid sequence of a human COX-2 polypeptide (SEQ ID NO:5).

FIG. 8 contains the nucleic acid sequence that encodes a human COX-2 polypeptide (SEQ ID NO:6).

### DETAILED DESCRIPTION

This document provides methods and materials for assessing the risk of developing breast cancer in patients with atypical hyperplasia. For example, this document provides methods and materials for using the level of Ki67 polypeptide expression in an atypical hyperplasia sample to determine if the patient with atypical hyperplasia has an increased risk of developing breast cancer within ten years of biopsy or diagnosis or an increased risk of developing breast cancer at a time point more than ten years after biopsy or diagnosis. The Ki-67 designation was originally for a mouse monoclonal antibody that recognizes a nuclear antigen present in cells that are actively proliferating (G1, S, G2, M), but is absent in resting cells (G0). The antigen recognized by the Ki-67 antibody is referred to as a Ki-67 polypeptide. MIB-1 is a commercially available antibody that is considered the reference monoclonal mouse antibody for the demonstration of the Ki-67 antigen in formalin-fixed, paraffin-embedded specimens.

As described herein, female atypical hyperplasia patients having atypical hyperplasia tissue containing an increased percentage of Ki67⁺cells (e.g., 2.0, 2.5, 3.0, or more percent Ki67⁺cells) or an increased intensity of Ki67⁺staining can be classified as having an increased risk of developing breast cancer during the first ten years after being diagnosed with atypical hyperplasia. Female atypical hyperplasia patients having atypical hyperplasia tissue containing a reduced percentage of Ki67⁺ cells (e.g., less than 2.0 percent Ki67⁺cells such as 1.9, 1.5, 1.0, 0.5, or less percent Ki67⁺cells) or a low intensity of Ki67⁺staining can be classified as having no increased risk of developing breast cancer (e.g., as compared to age-matched women without atypia) during the first ten years after being diagnosed with atypical hyperplasia and/or an increased risk of developing breast cancer after the first ten years from being diagnosed with atypical hyperplasia. As described herein, there is an association between higher Ki67 expression and higher COX-2 expression. Thus, in some cases, COX-2 expression can be used in combination with Ki67 expression or in place of Ki67 expression to assess the risk an atypical hyperplasia patient has for developing breast cancer as described herein.

The level of Ki67 expression and/or COX-2 expression can be determined using any appropriate method. For example, polypeptide detection methods such as cell or tissue staining, flow cytometry, immunohistochemistry, protein microarray assays, and Western blots can be used to determine the level of Ki67 polypeptide expression. In some cases, the level of Ki67 expression can be determined by determining the number or percentage of cells within a sample (e.g. an atypical hyperplasia tissue biopsy) that are Ki67⁺or by determining the intensity of Ki67 staining within a sample. Standard image analysis equipment can be used to determine the number or percentage of Ki67⁺cells as well as the intensity of Ki67 staining. In some cases, nucleic acid-based assays such as Northern blots, nucleic acid microarray assays, and RT-PCR can be used to assess the level of Ki67 expression by measuring the level of Ki67 mRNA within a sample.

A human Ki67 polypeptide can have the amino acid sequence set forth in SEQ ID NO:1 or 3 and can be encoded by the nucleic acid having the sequence set forth in SEQ ID NO:2 or 4. A human COX-2 polypeptide can have the amino acid sequence set forth in SEQ ID NO:5 and can be encoded by the nucleic acid having the sequence set forth in SEQ ID NO:6.

In general, a breast tissue biopsy sample can be obtained and used to evaluate the level of Ki67 and/or COX-2 expression. Any appropriate method can be used to obtain a sample. For example, a breast tissue sample can be obtained by standard breast tissue biopsy techniques. Once obtained, a sample can be manipulated prior to measuring the level of Ki67 and/or COX-2 expression. For example, a breast tissue sample can be treated such that total mRNA is obtained. Once obtained, the total mRNA can be evaluated to determine the level of Ki67 mRNA and/or COX-2 mRNA expression. In another example, a breast tissue sample can be a tissue sample that can be sliced into sections for immunostaining. Once obtained, the tissue slice can be analyzed using anti-Ki67 polypeptide antibodies (e.g., anti-Ki67 monoclonal antibodies such as MIB-1 commercially available from DAKO (clone M7240) and anti-Ki67[PP-67] antibody commercially available from Abcam (code ab6526)) and/or anti-COX-2 polypeptide antibodies (e.g., anti-COX-2 monoclonal antibodies such as those commercially available from DAKO; clone M3617, code number CX-294) to determine the level of Ki67 and/or COX-2 expression within the sample.

This document also provides methods and materials to assist medical or research professionals in determining the risk profile of a patient with atypical hyperplasia. Medical professionals can be, for example, doctors, nurses, medical laboratory technologists, and pharmacists. Research professionals can be, for example, principal investigators, research technicians, postdoctoral trainees, and graduate students. A professional can be assisted by (1) determining the level of Ki67 and/or COX-2 expression within a sample (e.g., an atypical hyperplasia sample), and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides methods and materials related to treating a human having atypical hyperplasia to reduce the risk of developing breast cancer. For example, a tissue biopsy can be obtained from a patient suspected to have a breast lesion or abnormality. After the tissue biopsy is examined using standard diagnostic techniques and determined to be an atypical hyperplasia, it can be assessed to determine the level of level of Ki67 and/or COX-2 expression as described herein. If the level is high (e.g., greater than 2.0 percent or more of the cells are Ki67⁺), then the patient can be treated with heightened surveillance or aggressive prophylactic treatment (e.g., tamoxifen, raloxifene, and/or prophylactic mastectomy) within the first few years of diagnosis (e.g., within one, two, or three years of diagnoses). If the level is low (e.g., less than 2.0 percent of the cells are Ki67⁺), then the patient can be instructed to pursue routine breast cancer surveillance during the first few years following diagnosis followed by a more aggressive prophylactic treatment (e.g., tamoxifen, raloxifene, and/or prophylactic mastectomy) as the patient approaches the ten year post-diagnosis mark.

The invention will be further described in the following examples, which do not limit the scope of the invention described in the claims.

### EXAMPLES

### Example 1

### The Expression Level of Ki67 Polypeptides is a Time-Varying Biomarker of Risk of Breast Cancer In Atypical Hyperplasia

### Study Population

The Mayo Benign Breast Disease cohort includes 9376 women ages 18-85 who had excisional breast biopsies with benign findings at the Mayo Clinic in Rochester, Minn. between Jan. 1, 1967 and Dec. 31, 1991. In this cohort, 331 women had atypical hyperplasia. Benign biopsies were reviewed by a study pathologists using current classification systems (Dupont and Page, N. Engl. J. Med., 312(3):146-51 (1985) and Page et al., Cancer, 55(11):2698-708 (1985)). Paraffin-embedded, formalin-fixed tissue for Ki67 staining was available for 192 of the women with atypia.

### Follow-Up and Risk Factor Data

Follow-up for breast cancer events and risk factor information were obtained through Mayo medical records and a study questionnaire. Family history was classified as negative, strong, or weak. Criteria for a strong family history were: at least one first-degree relative with breast cancer before the age of 50 years or two or more relatives with breast cancer, with at least one being a first degree relative. Any lesser degree of family history was considered to be weak.

### Histopathologic Review

A diagnosis of atypical ductal hyperplasia (ADH) or atypical lobular hyperplasia (ALH) was based on criteria described elsewhere (Page et al., Cancer, 55(11):2698-708 (1985)). For each example of atypical hyperplasia, the number of separate foci was defined (Degnim et al., J. Clin. Oncol., 25(19):2671-7 (2007)). Multifocal atypia required the identification of atypia in more than one terminal duct lobular unit, as defined by clear separation from another terminal duct lobular unit by nonspecialized interlobular mammary stroma (Degnim et al., J. Clin. Oncol., 25(19):2671-7 (2007)).

### Immunostaining

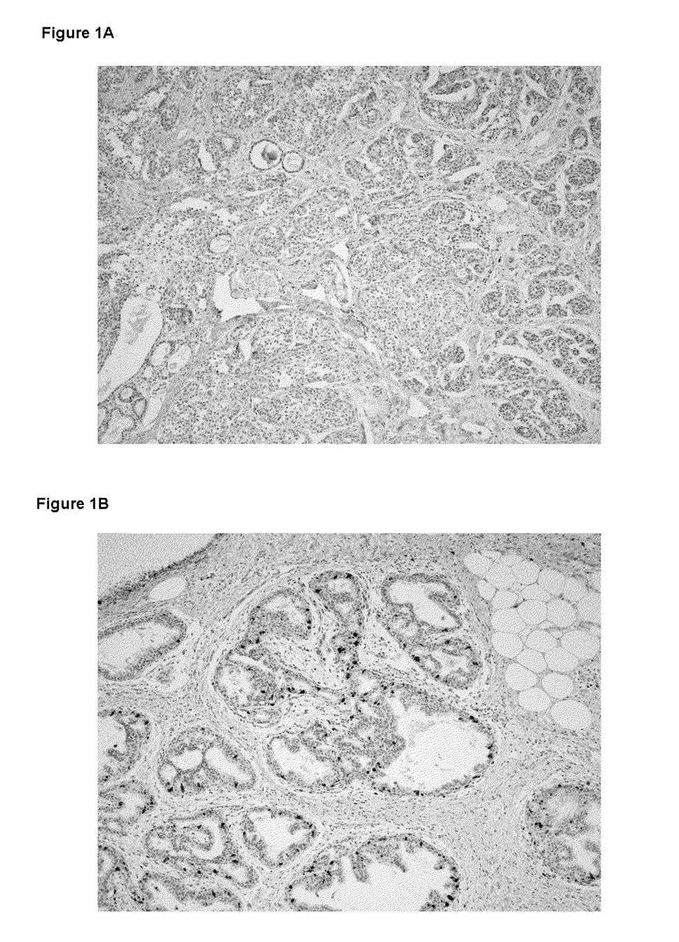

Ki67/MIB-1 assessment: Tumor proliferation was determined by digital image analysis with an Automated Cellular Imaging System (ACIS III®, DakoCytomation, San Juan Capistrano, Calif.) using 5 μm-thick formalin-fixed paraffin-embedded sections. All analyses were performed in the Molecular Imaging Laboratory within the Department of Pathology at Mayo Clinic. Ki67 expression was assessed with the nuclear antibody MIB-1 (Immunotech, Marseille, France; 1:400 dilution) using a standard avidin-biotin complex method. Slides were processed with an automatic stainer (BioTek®, Ventana Medical System, Tucson, Ariz.), and diluted hematoxylin was used as a nuclear counterstain. Nuclei expressing the antigen that reacts with Ki67/MIB-1 were identified by the brown chromogen diaminobenzidine. Representative Ki67 staining is provided in FIG. 1.

Areas of atypia were marked as ADH or ALH on each slide by a pathologist. Each slide was scanned on the ACIS III to generate an image for quantitative analysis. On each image, all areas of atypical epithelium (stroma was excluded) as marked by the pathologist were outlined using the freeform tool for analysis by the software. The instrument provided a Ki67/MIB-1 percentage (representing the proportion of the nuclear area positive for MIB-1) and intensity (representing the average stain concentration over the region collected on a scale of 0 to 255) for each specimen. For women with more than one area of atypia, per-woman summary measures of percent staining were calculated using a weighted average of the multiple values, with weights proportional to the size of each given area. Summary measures of intensity for each woman were calculated in a similar fashion, but used both area-size and area-specific percent staining as weighting variables to account for potential differences in these factors across the multiple areas per women.

### Statistical Analysis

Data were summarized descriptively using frequencies and percentages for categorical variables and medians and interquartile ranges (IQRs) for continuous variables. Among the 331 women with atypical hyperplasia, distributions of demographic and clinical variables by tissue availability for Ki67 staining were compared using chi-square tests of significance.

The length of follow-up for each woman in the study was defined as the number of days from her benign biopsy to the date of her breast cancer diagnosis, prophylactic mastectomy, LCIS, death, or last contact. Relative risks, overall and by strata of Ki67 staining levels, were estimated with standardized incidence ratios (SIRs) by dividing the observed numbers of incident breast cancer by population-based expected values. The approach allowed one to compare rates of breast cancer in the cohort with that of the general population rather than an internal referent group, recognizing that all women in the cohort were at some increased risk of breast cancer from their diagnosis of atypical hyperplasia. Expected values were calculated by apportioning each woman's person-years of follow-up into 5-year age and calendar-period categories and multiplying these by the corresponding breast cancer incidence rates from the Iowa Surveillance, Epidemiology, and End Results (SEER) registry. This reference population was chosen because of its demographic similarities to the Mayo Clinic population (80% of cohort members reside in the upper Midwest). Risk analyses were performed within two time-dependent strata, one that focused on follow-up and outcomes that occurred in the first 10 years post biopsy and one that focused exclusively on follow-up and outcomes that occurred after the first 10 years post biopsy. Differences between the standardized incidence ratios across Ki67 staining levels were compared using a Poisson regression analysis that accounted for the population-based expected event rate for each individual using an offset term. Separate analyses were carried out for percent staining and intensity of staining.

Observed and expected event rates were visually displayed using cumulative incidence curves, while the effects of death as a competing risk were taken into account (Gooley et al., Stat. Med., 18(6):695-706 (1999)). Expected events were calculated for each one-year follow-up interval in a manner similar to that used for determining SIRS. A modified Kaplan-Meier approach was used to accumulate expected incidence over these intervals. The expected curve was smoothed using linear interpolation. All statistical tests were two-sided, and all analyses were conducted using the SAS (SAS Institute, Inc., Cary, N.C.) software system.

### Results

### Patient Characteristics

Within the atypia cohort of 331 women, archived tissue was available for Ki67 staining for 192 women. These 192 subjects were compared to the remaining 139, and no significant differences were observed in terms of distributions of case status, age at atypia, family history of breast cancer, and for breast cancer patients, time to diagnosis (p>0.05 for each attribute). Clinical features for the subjects included in the study are presented in Table 1, along with histopathologic features of the atypias. Median post-biopsy follow-up was 14.6 years for the 192 women, 32 of whom (16.7%) have developed breast cancer.

TABLE 1
Clinical, histologic, and molecular features by
levels of Ki-67 staining

| | <2% Ki67⁺ | 2+% Ki67⁺ | |
|---|---|---|---|
| | cells | cells | |
| | (N = 134) | (N = 58) | p values¹ |
| Age at BBD diagnosis | | | |
| <30 | 0 (0%) | 1 (100%) | 0.20 |
| 30-39 | 5 (62.5%) | 3 (37.5%) | |
| 40-49 | 38 (82.6%) | 8 (17.4%) | |
| 50-59 | 34 (69.4%) | 15 (30.6%) | |
| 60-69 | 30 (66.7%) | 15 (33.3%) | |
| 70+ | 27 (62.8%) | 16 (37.2%) | |
| Foci | | | |
| 1 Focus | 75 (72.8%) | 28 (27.2%) | 0.33 |
| 2+ Foci | 59 (66.3%) | 30 (33.7%) | |
| Family History | | | |
| Missing | 8 | 3 | 0.10 |
| None | 68 (64.2%) | 38 (35.8%) | |
| Weak | 29 (82.9%) | 6 (17.1%) | |
| Strong | 29 (72.5%) | 11 (27.5%) | |
| Type of Atypia | | | |
| ADH | 47 (69.1%) | 21 (30.9%) | 0.54 |
| ALH | 67 (72.8%) | 25 (27.2%) | |
| Both ADH and ALH | 20 (62.5%) | 12 (37.5%) | |
| COX-2 Staining* | | | |
| 0-1+ | 69 (77.5%) | 20 (22.5%) | 0.03 |
| 2-3+ | 53 (62.4%) | 32 (37.6%) | |
| Follow-Up in Years | | | |
| Median (IQR) | 14.6 (9.4-18.8) | 14.6 (8.9-18.0) | 0.39 |

¹Categorial variable p-values from chi-square tests; continuous variable p-values from Wilcoxon rank sum test
*Available for 174 individuals (Visscher et al., J. Natl. Cancer Inst., 100(6): 421-7 (2008))

### Ki67 Immunostaining of Atypia Samples

In the 192 samples, both percent cells positive for Ki67 and staining intensity were analyzed. The correlation between the two measures was 0.98. Thus, the values are reported by percent cells positive, as is customary when reporting Ki67 values. The median value for percent positive cells for Ki67 was 1.0%; the 75th percentile value was 2.3%. Based on the empirical distribution of staining values in the cohort, a cutoff of 2% cells positive was selected to define groups of individuals with higher or lower degrees of staining. Examples of low (<2%) and high (>2%) staining of Ki67 are shown in FIG. 1. The results were examined for patterns in Ki67 levels by age at biopsy, type of atypia (ADH, ALH, or both), number of foci of atypia, and family history. No differences were observed as shown in Table 1. For 174 of these 192 women, COX-2 staining data was obtained (Visscher et al., J. Natl. Cancer Inst., 100(6):421-7 (2008)). There was an association between higher Ki67 expression and higher COX-2 expression (p=0.03).

Association of Ki67 Expression with Breast Cancer Risk

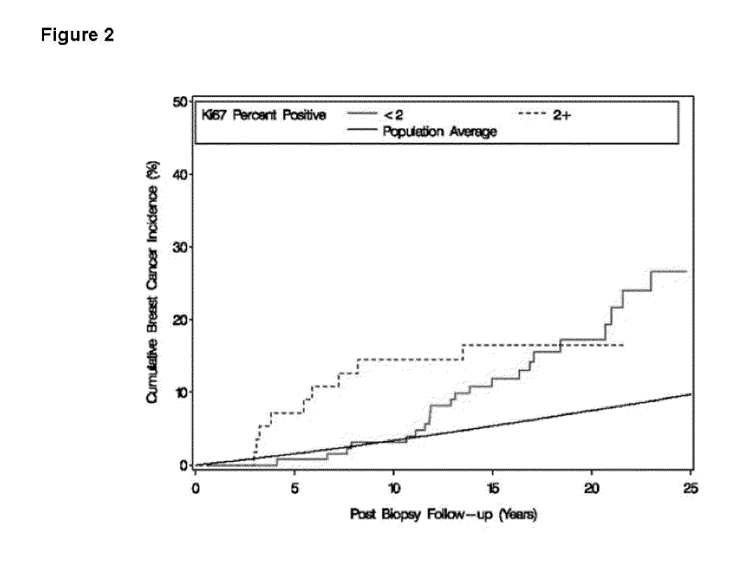

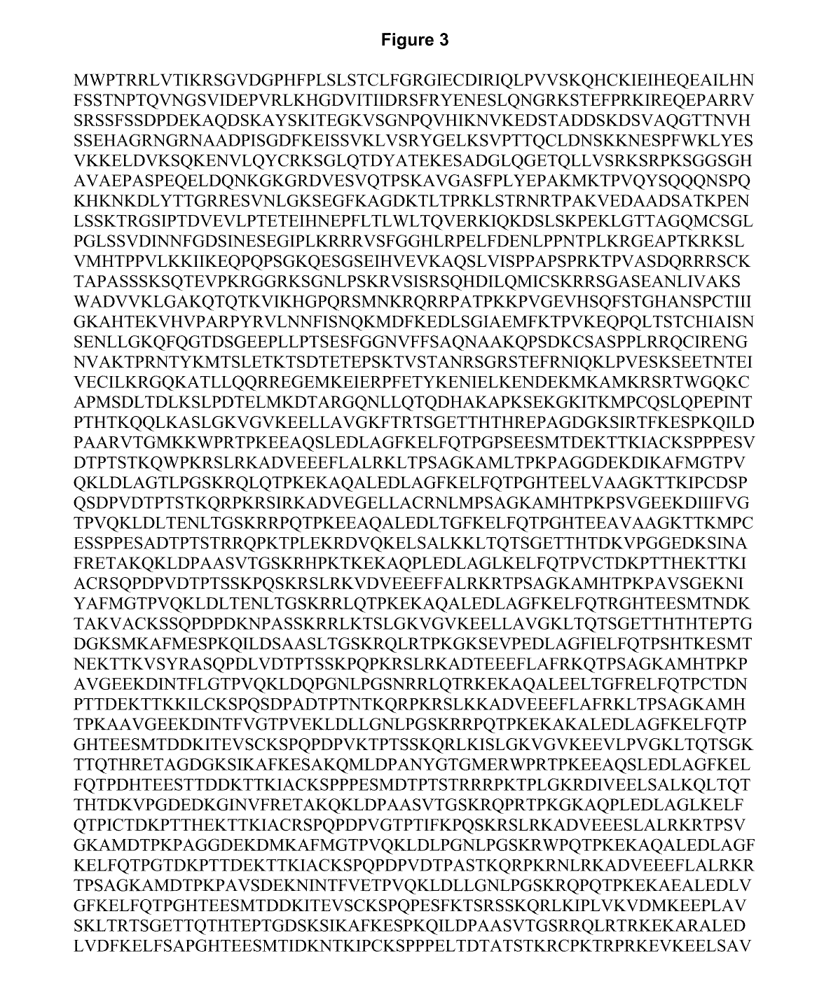

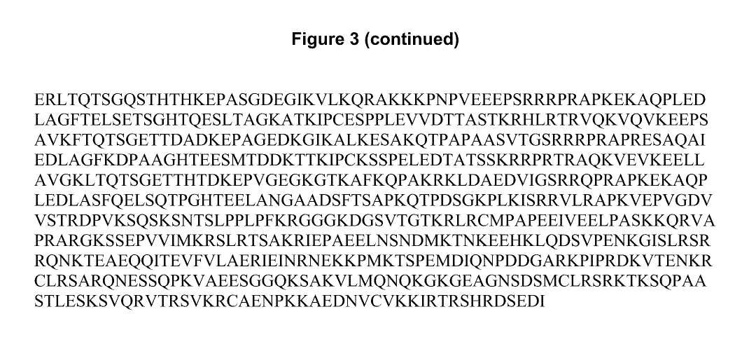

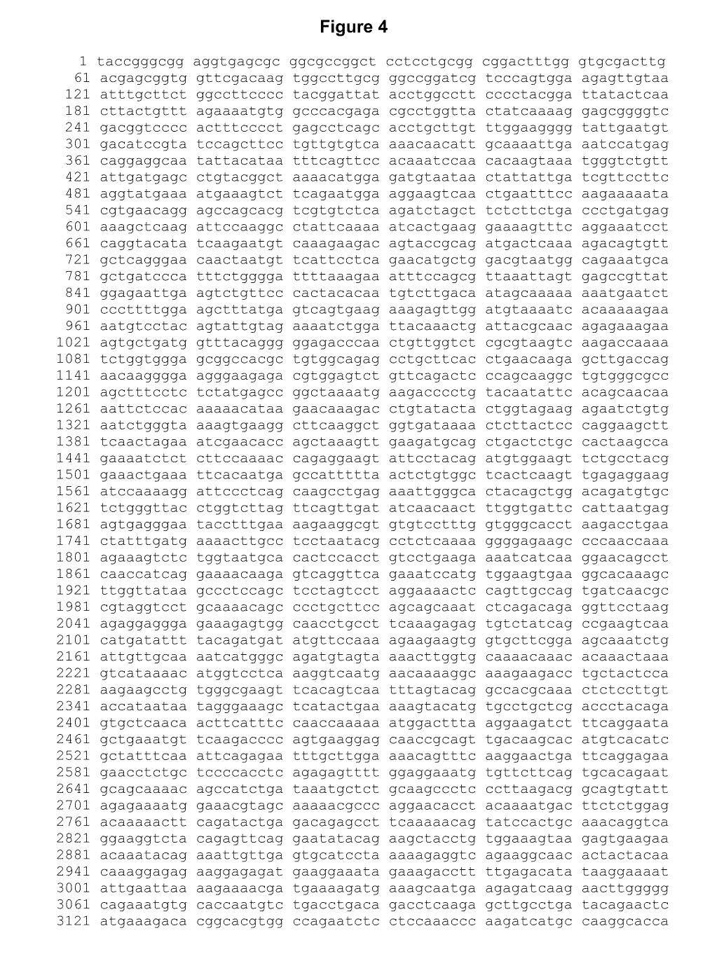

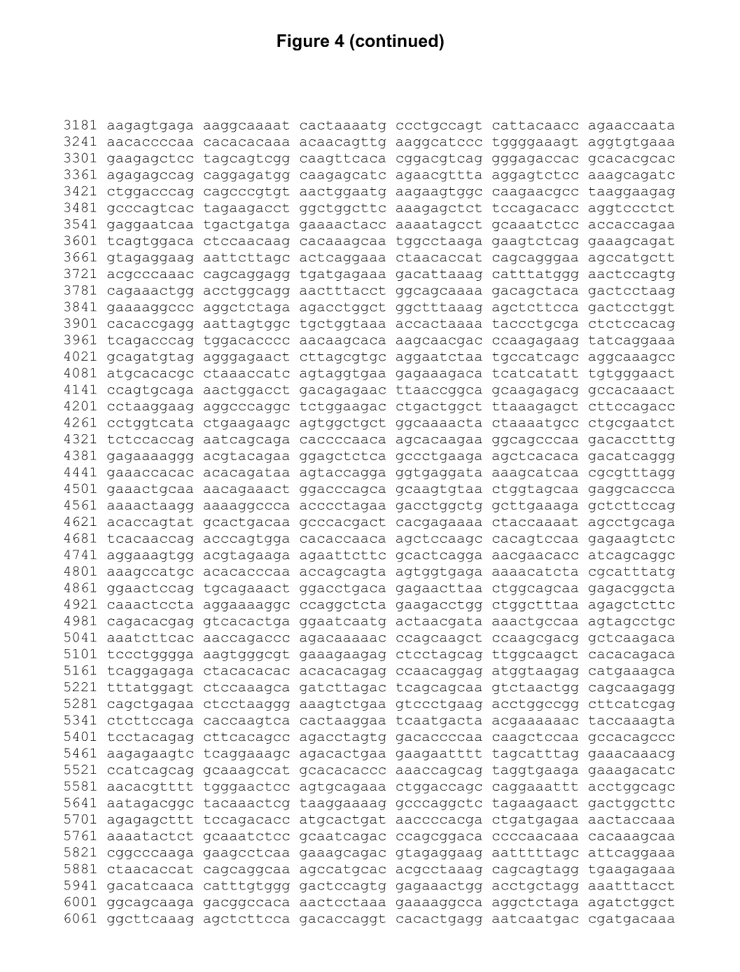

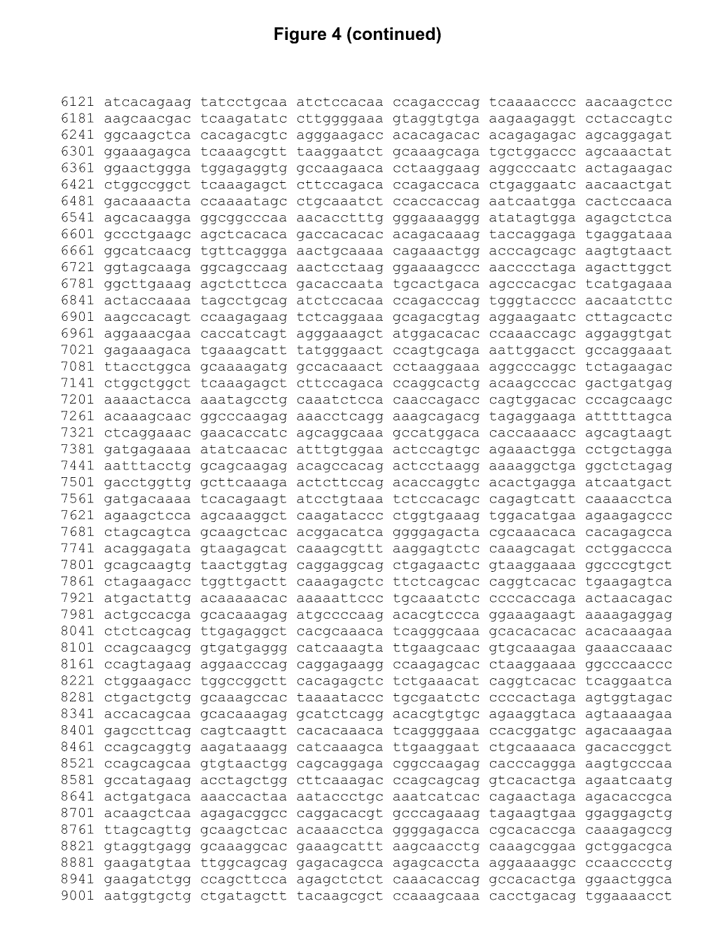

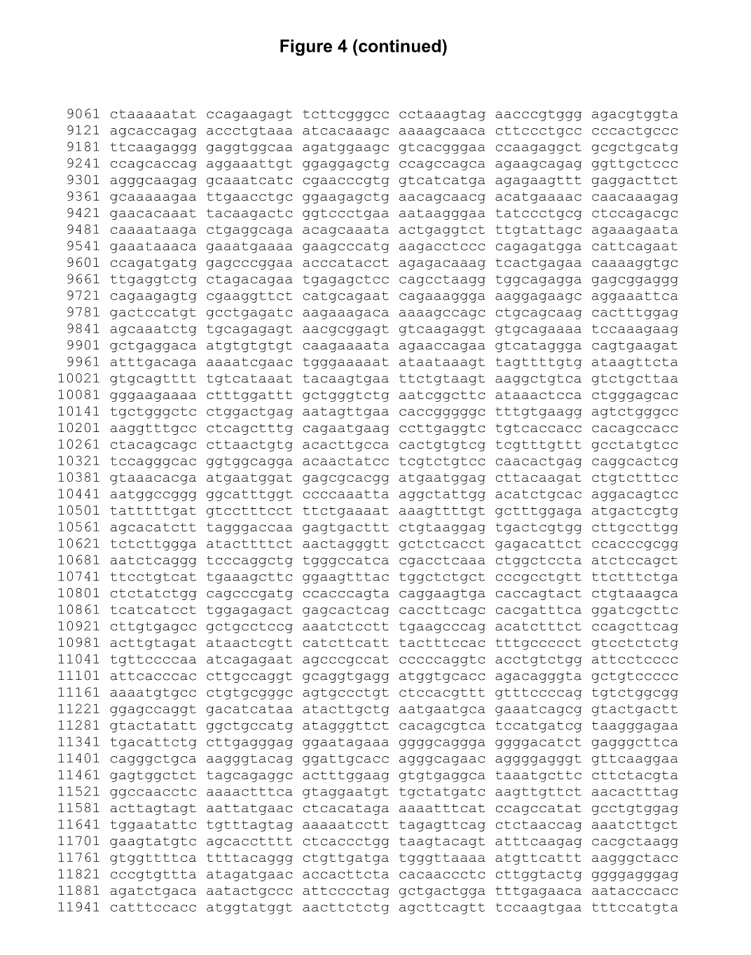

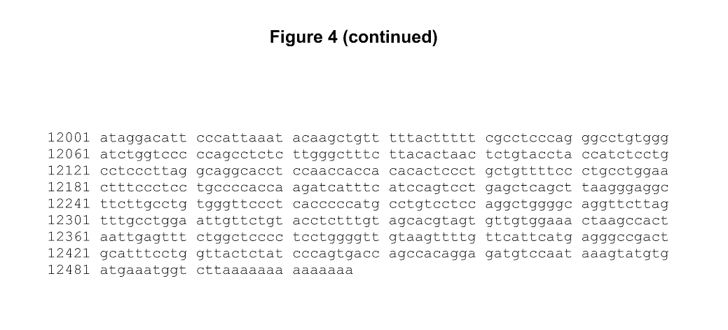

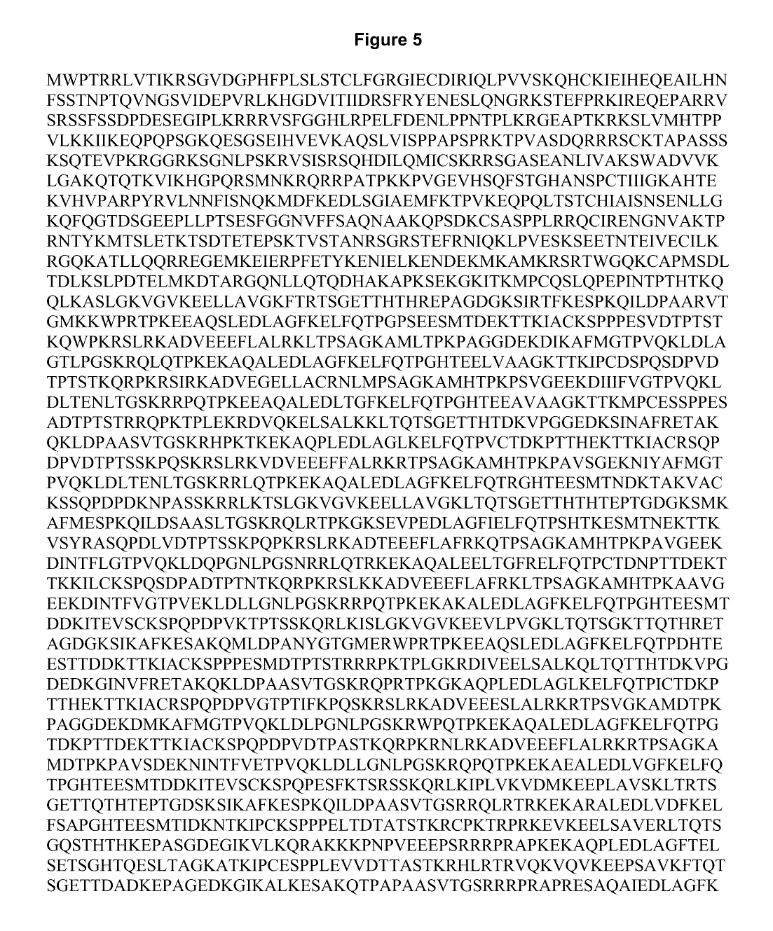

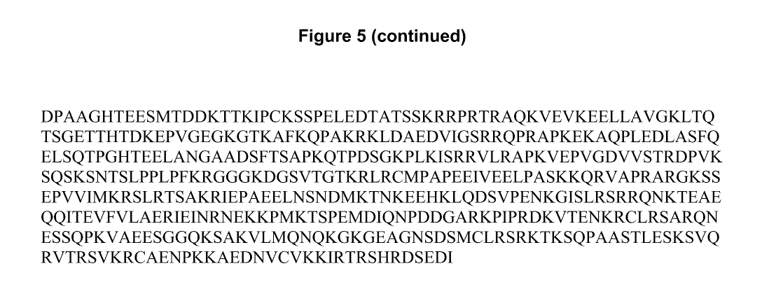

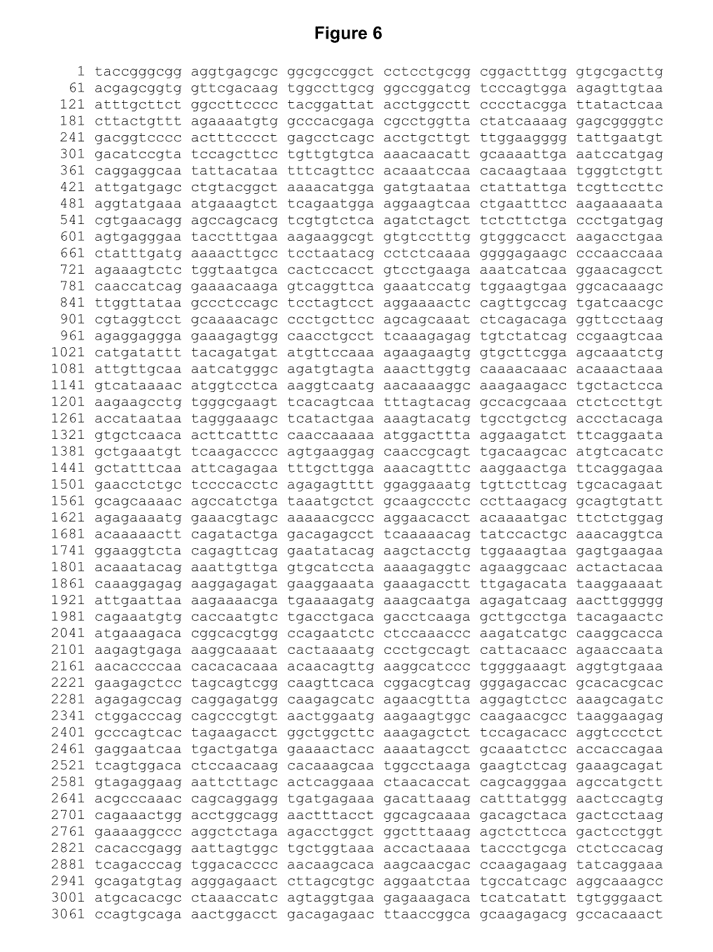

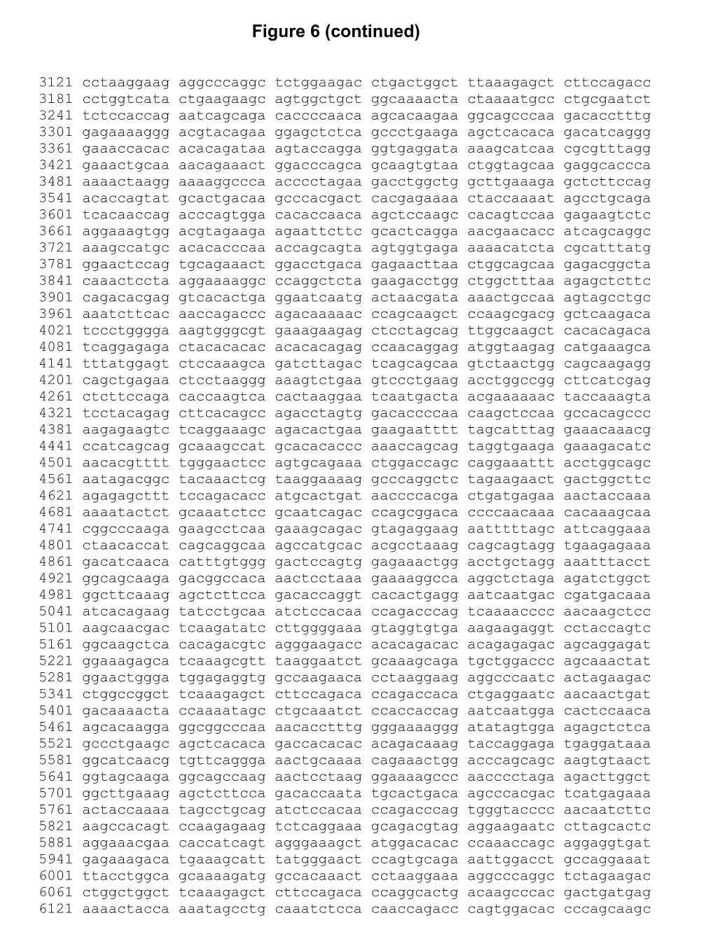

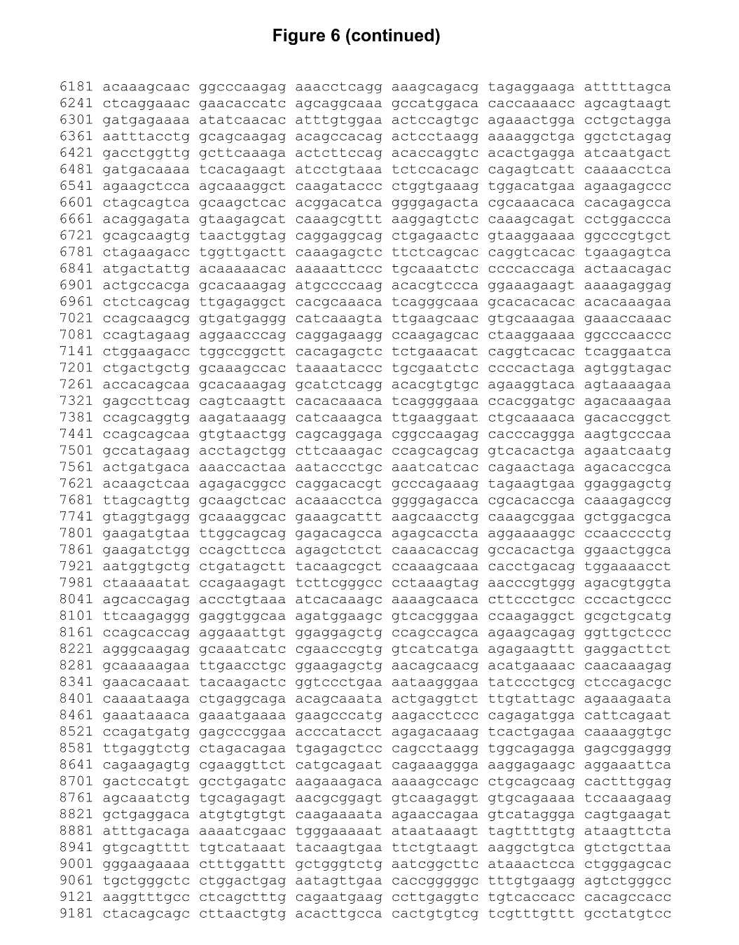

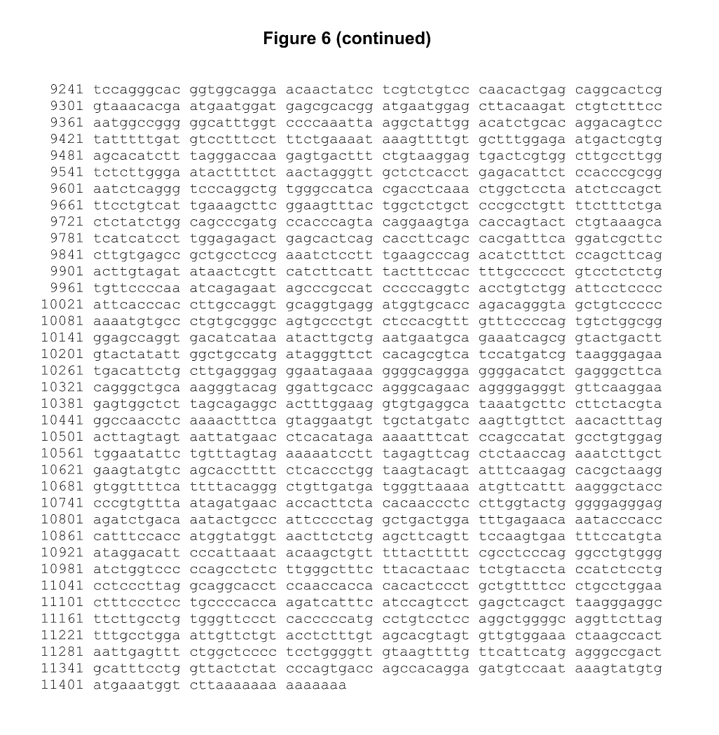

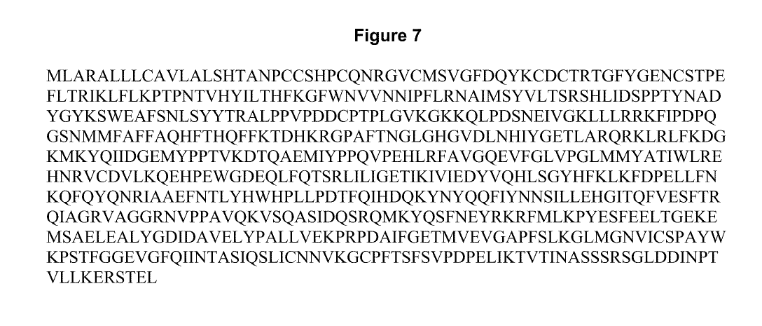

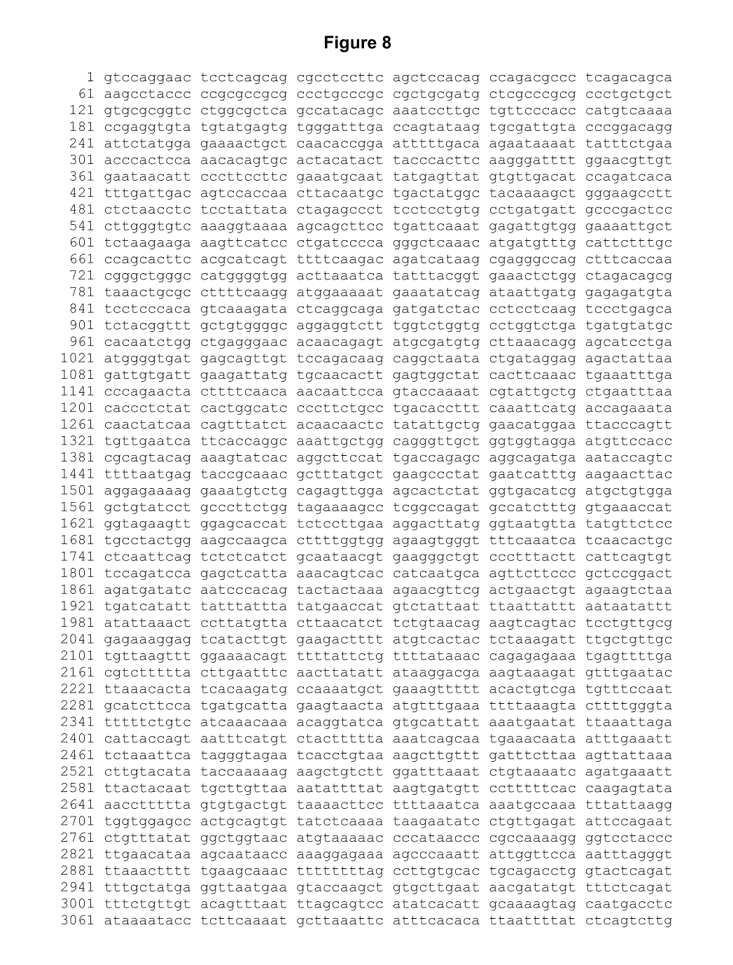

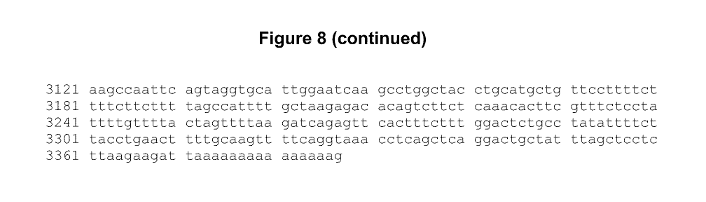

Among cases, the time from atypia biopsy to breast cancer diagnosis was examined by Ki67 immunostaining. Among the 32 patients who developed breast cancer, those with ≧2% cells staining positive for Ki67 expression had a shorter time to breast cancer (median=5.5 years, IQR=3.2-7.2) than those with <2% cells staining positive for Ki67 (median=13.8 years, IQR=11.6-20.3). This difference was reflected in the results displayed in Table 2 and FIG. 2. The risk of breast cancer was analyzed by Ki67 levels within two time periods: over the first 10 years of follow-up after diagnosis of atypia and in the follow-up after 10 years. As shown in Table 2, a positive association was found between Ki67 overexpression (≧2% of cells positive) and the risk of breast cancer in the first 10 years of follow-up (SIR=4.42 [95% CI 2.21-8.84]). This excess risk resulted in a 10-year cumulative incidence of 14.1%, which is significantly higher than what is expected in the population at large (FIG. 2). In contrast, in the women with low Ki67 expression, no increased risk of breast cancer was found in the first 10 years, with SIR 1.01 (95% CI 0.38-2.70), which was significantly lower than the women with high expression (p=0.01). The 10-year cumulative incidence of breast cancer was 3% for those women with low Ki-67 staining values, in line with population averages. After 10 years, risk increased significantly in the low Ki67 group [SIR 5.69 (3.63-8.92)] vs. no increased risk in the high Ki67 group [SIR 0.78 (0.11-5.55)]. This apparent time-dependent difference in incidence patterns by Ki67 staining levels was formally examined via Poisson regression analysis and found to be a statistically significant interaction between pre-vs. post-10 years incidence and high vs. low staining levels (p<0.001).

TABLE 2
Risk of breast cancer in a typical hyperplasia by Ki67 expression and follow-up interval.

| Ki67 percent | | Ten-year risk | | | | Post 10-year risk | | | |
|---|---|---|---|---|---|---|---|---|---|
| cells stained | | Observed | Expected | SIR | | Observed | Expected | SIR | |
| positive | N | events | events | (65% CI)¹ | p-value² | events | events | (95% CI)¹ | p-value² |
| <2% | 134 | 4 | 3.9 | 1.01 | 0.01 | 19 | 3.3 | 5.69 | 0.008 |
| | | | | (0.38-2.70) | | | | (3.63-8.92) | |
| ≧2% | 58 | 8 | 1.8 | 4.42 | | 1 | 1.3 | 0.78 | |
| | | | | (2.21-8.84) | | | | (0.11-5.55) | |

¹Standardized incidence ratios and corresponding 95% confidence intervals, comparing the observed number of breast cancer events to those expected based on incidence rates from Iowa SEER data. Analyses account for the effects of age and calendar period.
²Test for heterogeneity of the standardized incidence ratio across staining levels.

Primary analyses were based on a Ki67 staining cutpoint of 2%. However, a defined standard of high staining values for Ki67 in atypia has yet to be established. Thus, the associations were re-examined using different cutpoints ranging from 0.5 to 3% and similar results were obtained.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining an atypical hyperplasia patient's time period of increased risk for developing breast cancer, wherein said method comprises determining if an atypical hyperplasia sample obtained from a patient contains an increased level of Ki67 or COX-2 expression, classifying said patient as having an increased risk of developing breast cancer within an early phase from diagnosis if said atypical hyperplasia sample contains said increased level, and classifying said patient as having an increased risk of developing breast cancer within a late phase from diagnosis if said atypical hyperplasia sample does not contain said increased level.

2. The method of claim 1, wherein said sample is a tissue section.

3. The method of claim 1, wherein said method comprises determining if said sample contains an increased level of Ki67 expression.

4. The method of claim 3, wherein said increased level of Ki67 expression is greater than 2.0 percent Ki67⁺cells within said sample.

5. The method of claim 1, wherein said method comprises determining if said sample contains an increased level of COX-2 expression.

6. The method of claim 1, wherein said method comprises determining if said sample contains an increased level of Ki67 or COX-2 expression using an anti-Ki67 antibody or an anti-COX-2 antibody.

7. The method of claim 1, wherein said method comprises determining if said sample contains an increased level of Ki67 or COX-2 expression using a nucleic acid detection method.

8. The method of claim 1, wherein said early phase from diagnosis is within eight years of diagnosis.

9. The method of claim 1, wherein said early phase from diagnosis is within nine years of diagnosis.

10. The method of claim 1, wherein said early phase from diagnosis is within ten years of diagnosis.

11. The method of claim 1, wherein said late phase from diagnosis is the time after ten years from diagnosis.
